(19) United States
(12) Patent Application Publication
(10) Pub. No.: US 2021/0285905 A1
(43) Pub. Date: Sep. 16, 2021

(54) Method and Device for Measuring Dielectrics in Fluids

(71) Applicant: Stichting Wetsus Intellectual Property Foundation, Leeuwarden (NL)

(72) Inventors: Jorrit Hillebrand, Leeuwarden (NL); Mateo Jozef Jacques Mayer, Leeuwarden (NL); Gerardus Cornelis Maria Meijer, Leeuwarden (NL); Louis Cornelia Patrick Maria De Smet, Leeuwarden (NL); Ernst Jan Robert Sudhölter, Leeuwarden (NL)

(21) Appl. No.: 16/615,528

(22) PCT Filed: May 24, 2018
(86) PCT No.: PCT/NL2018/050343

§ 371 Date: Nov. 21, 2019

(30) Foreign Application Priority Data
May 24, 2017 (NL) 1042400

(51) Int. Cl. G01N 27/02; G01N 33/18

(52) U.S. Cl. CPC G01N 27/026; G01N 33/18

(57) ABSTRACT

The present invention relates to a method and device for measuring dielectrics in fluids, such as water. The sensor according to the invention comprises: a first printed circuit board (PCB) having a first side and a second side; a first and second conductor on a side of said first PCB; at least one galvanic connection between the first conductor and the second conductor; a first and second polymer affinity layer on a side of the first PCB; a second and third PCB, being equipped with holes and with a conductor plate; at least one galvanic connection between the first conductor plate and the second conductor plate; at least a first function generator; and a first spectrum analyzer or rectifier, wherein, in use, the sensor is placed in the fluid, and fluid contacts the polymer affinity layer through the holes and wherein impurities in the fluid are selectively absorbed and dispensed in the polymer layers thereby changing the dielectric properties of the polymer affinity layers.

The present invention relates to a method and device for measuring dielectrics in fluids, such as water.

Nowadays, the main way of measuring contaminants in water is grab-sampling, i.e. sending the samples to a lab and waiting for the results. The use of conventional measuring devices is limited because of parasitic effects, range limitations (frequency and concentration) and/or sensitivity to defects.

The object of the present invention is to provide a sensor that obviates or at least reduces the aforementioned problems.

The objective is achieved with the sensor according to the invention, the sensor comprising:
a first printed circuit board (PCB) having a first side and a second side;
a first conductor on the first side of said first PCB;
a second conductor on the second side of said first PCB;
at least one galvanic connection between the first conductor and the second conductor;
a first polymer affinity layer on the first side of the first PCB;
a second polymer affinity layer on the second side of the first PCB;
a second PCB, wherein the second PCB is equipped with holes and with a first conductor plate, wherein the second PCB is placed on top of the first polymer affinity layer;
a third PCB, wherein the third PCB is equipped with holes and with a second conductor plate, wherein the second PCB is placed on top of the second polymer affinity layer;
at least one galvanic connection between the first conductor plate and the second conductor plate;
at least a first function generator that is operatively connected to the first and second conductors as well as to the first and second conductor plates; and
at least a first spectrum analyzer or rectifier that operatively connected to the first and second conductors as well as to the first and second conductor plate for measuring the amplitude of the electrical signal transmitted from the function generator through the measuring system;

wherein, in use, the sensor is placed in the fluid and fluid contacts the polymer affinity layer through the holes and wherein impurities in the fluid are selectively absorbed and dispensed in the polymer layers thereby changing the dielectric properties of the polymer affinity layers.

The sensor according to the present invention comprises a first PCB that is more or less sandwiched between the first and second polymer affinity layers and between the second and third PCBs. In use, the sensor is placed in the fluid under investigation and the polymer affinity layers in the sensor absorb chemical compounds and/or ions present in the fluid. Since the sensor behaves electrically as a stub resonator, the absorbed compounds and/or ions can be preferably characterized or identified through impedance spectroscopy.

An advantage of the sensor according to the present invention is that is enables a sensor that can be placed in-line and read-out remotely, giving the analysis results real-time.

Furthermore, an advantageous aspect of the present invention concerns the geometry of the device. The proposed geometry ensures that essentially all electric field lines run through the affinity layer. Prevention of stray field lines (i.e. lines reaching out of the affinity layer) to surrounding dielectric and conductive material makes the sensor insensitive to changing dielectric conditions outside the affinity layer. By implication, the sensitivity of the sensor solely depends on the change of the dielectric properties accomplished by the targeted analyte that has been absorbed by the affinity layer. Fine tuning the chemistry of the affinity layer allows the design of sensors with different affinities for different analytes. This opens possibilities for designing a product range of sensors with selectivity for different analytes. The first and second polymer affinity layer may be manufactured of an identical material or of different materials wherein the chosen materials preferably depends on the analyte(s) to be detected.

The sensor according to the present invention has as a further advantageous effect that it is easy to manufacture. For example, the first, second and third PCBs can be produced using standard PCB manufacturing techniques. The PCBs can be immobilized and connected easily through connectors such as plastic screws. Alternatively or additionally, spacers, such as plastic beads or glass beads, can be put between the PCBs so that there is a uniform distance between them. Subsequently, the PCBs can be placed in a mould and the polymer affinity layer can be poured into the mould. After cross linking and electrically connecting the sensor, it is ready for use. This method of production makes expressly part of the technology according to the present invention.

In a presently preferred embodiment of the invention the first and second conductors of the first PCB comprise a meandering pattern over the PCB surface, thereby increasing the effective length of said conductors.

From a practical point of view, it may be desirable to produce a sensor with a relatively low resonant frequency. Since the resonant frequency decreases with increasing length of the conductors on the first PCB, a low resonant frequency of the sensor may require unacceptably long PCBs. It is noted that this problem can be overcome by application of meander conductors on both sides of the first PCB.

In a presently preferred embodiment of the invention the sensor further comprises guard electrodes.

Preferably, a width of the guard electrodes is at least 3 time a thickness of the affinity layers.

In an embodiment of the invention the guard electrodes comprise galvanically connected electrodes, wherein the guard electrodes are preferably grounded.

In an embodiment of the invention the guard electrodes are held at a fixed potential and are not galvanically connected to the electrodes.

In an embodiment of the invention the guard electrodes and the conductors are spaced apart at a predetermined distance.

In a presently preferred embodiment of the invention the sensor is configured for one or more of: detection of oil traces in water, detection of metal ions in water, detection of medicine traces in water, detection of pesticides in water, detection of traces of drugs in water e.g., narcotics, XTC and cocaine, detection of detergents in water including non-ionic detergents e.g., pentaerythrityl palmitate and ionic detergents e.g., sodium dodecyl sulfate, and for detection of nutrients in water such as phosphates, nitrates and sulfates.

The present invention further relates to a method for measuring dielectric properties of chemical compounds and/or ions in water, comprising the step of providing a sensor in an embodiment of the present invention.

The method provides the same effects and advantages as described for the sensor.

Further advantages, features or details of the invention are elucidated on the basis of preferred embodiments thereof, wherein reference is made to the accompanying drawings, in which:

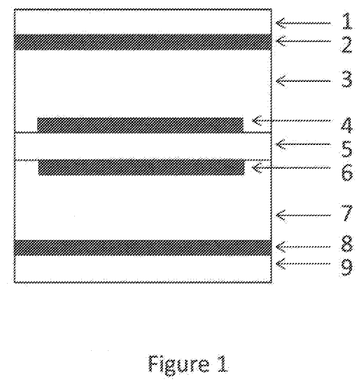

FIG. 1 shows an embodiment of the sensor according to the invention;

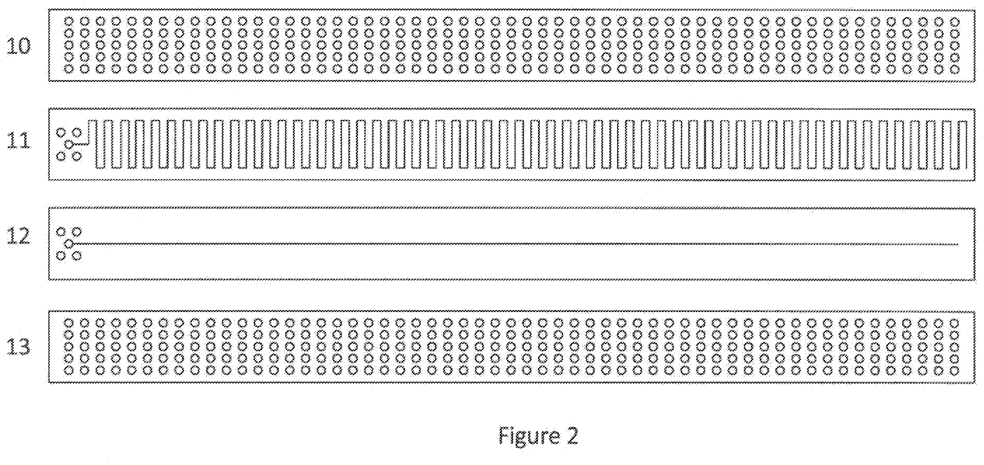

FIG. 2 shown an embodiment of a PCB design;

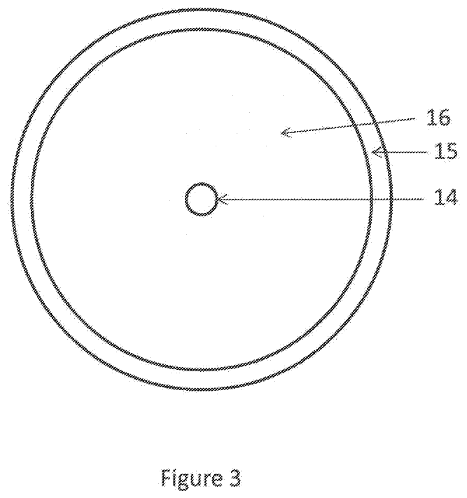

FIG. 3 gives a schematic overview of a coaxial transmission line in an embodiment of the invention;

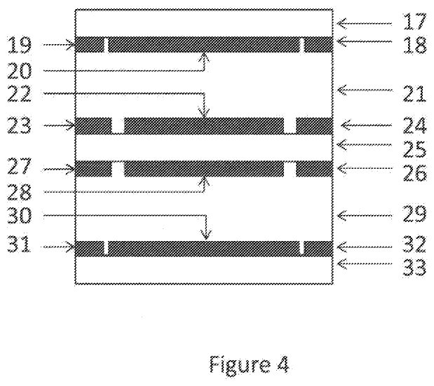

FIG. 4 shows a cross section of a sensor similar to the sensor described in FIG. 1; and

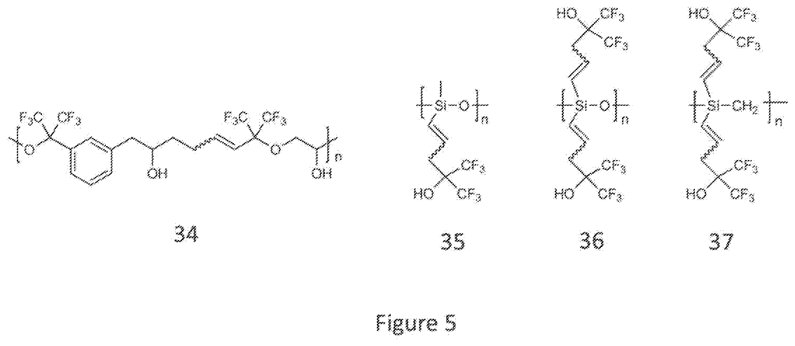

FIG. 5 shows some molecular structures.

The present invention relates to a first, preferably rectangular PCB, indicated in the cross section perpendicular to the length axis of the sensor in FIG. 1 with numbers 4, 5 and 6. The first PCB is equipped with two, preferably identical, conductors 4 and 6 that are present on each side of said first PCB. The geometry of conductors 4 and 6 is preferably identical and their position on the first PCB is preferably such that both conductors are exactly on top of each other with the first PCB substrate in between. Preferably, both conductors 4 and 6 on each side of the first PCB are galvanically connected, either through one connection point or more preferably through a series of vias along the length coordinate of the first PCB. In this patent application, a via is defined as a metal-plated hole in a PCB, galvanically connecting a conductor on one side of the PCB to a conductor on the other side of the PCB. This design of the first PCB results in one electrode that can be applied in a design of two parallel capacitors, behaving like a stub resonator at high frequencies, without the substrate dielectric 5 of the first PCB influencing the dielectric properties of said two parallel capacitors. This will be further explained later in the text of this patent application.

In FIG. 2, the PCB design indicated with number 12 shows a preferred embodiment of a first PCB. At the left side of PCB number 12, the connection points of an SMA connector can be seen. It is noted that, obviously, these contacts can also be used for making wire connections and also that other kinds of electrical connections can be made. The straight line at the center of the PCB with number 12 (FIG. 2) shows conductor 4 in FIG. 1 located on the top side of the first PCB. There is also a conductor 6 at the bottom side of the first PCB. Both conductors 4 and 6 are positioned at the center of the first PCB, exactly above each other with the substrate 5 of the first PCB in between. Both conductors 4 and 6 are galvanically connected at the SMA connection point at the left side of PCB number 12 in FIG. 2. It is noted that, more preferably, both conductors 4 and 6 on each side of the first PCB are interconnected through vias along the length coordinate of the first PCB. These vias are not depicted in FIG. 2.

According to a further embodiment of the invention, a second PCB, is equipped with holes and a first conductor plate 2, placed on top of the first affinity layer 3 and a third PCB, equipped with holes and a second conductor plate 8, placed below the second affinity layer 7. In FIG. 1, the numbers 1, 9 and 2, 8 indicate the substrates (numbers 1 and 9) and the conductor plates (numbers 2 and 8) of both preferably identical, second and third PCBs respectively. In FIG. 2, the rectangular PCBs, indicated with numbers 10 and number 13, show practical examples of second and third PCBs. It is noted that the conductor plates of the second and third PCBs are not shown on the perforated PCBs with number 10 and 13 in FIG. 2. Further, it is noted that the conductor plates 2 and 8 of the second and third PCBs have holes at exactly the same spot as the holes in the substrate of the second and third PCBs. Finally, it is noted that the conductor plates 2 and 8 of the second and third PCB are galvanically connected through connectors and/or wires and/or any other conductor. The result is that the first PCB and the second PCB form a first capacitor and that the first PCB and the third PCB form a second capacitor. It is noted that, from an electrical point of view, the first and second capacitors are positioned in parallel.

After describing the different aspects of the present invention, the technology according to the present invention will now be further explained. It is noted that, in this patent application, the term PCB stands for a printed circuit board comprising both a support layer (preferably FR4 material) and any conductors on this support layer.

In a presently preferred embodiment of the invention, the sensor in FIG. 1 comprises the electrical equivalent of two capacitors in parallel: A first capacitor formed by the first PCB and the second PCB with the polymer affinity layer 3 as dielectric in between and a second capacitor formed by the first PCB and the third PCB with the polymer affinity layer 7 as dielectric in between.

In use, a sensor according to FIG. 1 is placed in water containing impurities that are selectively absorbed by polymer affinity layers 3 and 7. Placing the sensor in FIG. 1 in water will result in contact between the water and the polymer affinity layers 3 and 7, mainly through the holes in the second and third PCBs. As a result, the polymer affinity layer will selectively absorb the impurities in the water. As a result of diffusion, the impurities will be distributed over the polymer affinity layers 3 and 7 thereby changing the dielectric properties of these layers. Because of the very specific design of the sensor in FIG. 1, the capacitance of the sensor will only change because of changing dielectric properties of polymer affinity layer 3 and not directly because of a change in the dielectric properties of the water in which the sensor is placed. Additionally, the dielectric properties of the substrate of the first, second and third PCBs do not significantly influence the capacitance of the sensor, thereby making it more sensitive as compared to designs where the sensor capacitance is also a function of the dielectric properties of the PCB substrate. This property of the sensor is caused by its geometry which is such that all the electrical field lines go through polymer affinity layer 3. As a result, the sensor according to the present invention is very feasible as inline sensor i.e., it can be placed in the fluid to be investigated.

In a preferred embodiment, the sensor according to FIG. 1 is placed in a first housing e.g., a cylinder or another water container with a fluid inlet and a fluid outlet. The fluid under investigation is pumped through the first housing such that it flows along the holes in the second and third PCBs. As a result, there is fast mass transfer of impurities in the fluid to the polymer affinity layers 3 and 7, thereby reducing the response time of the sensor.

As already indicated, the sensor can be applied as an inline capacitance sensor to detect changes in water quality and the chemical composition of aqueous solutions.

In a further embodiment of the invention the sensor is used for for impedance spectroscopy. The geometry of the sensor in FIG. 1 is such that this sensor is an electrical equivalent of a piece of coaxial transmission line.

FIG. 3 gives a schematic overview of a coaxial transmission line. In FIG. 3, the number 14 indicates the inner conductor of the coaxial transmission line, the number 15 indicates the outer conductor of the coaxial transmission line and the number 16 indicates the dielectric of the coaxial transmission line, usually a polymer. All field lines between the inner conductor 14 and the outer conductor 15 go through dielectric 16. A piece of transmission line is known to have a capacitance, an inductance and to behave like a stub resonator. This property makes transmission line based sensors very feasible for impedance spectroscopy i.e., for studying the properties of a dielectric 2 as a function of frequency. As a result, not only the static capacitance of the dielectric, but also its dielectric losses as a function of frequency can be studied.

Analogous to FIG. 3, the sensor design in FIG. 1, comprising PCBs with numbers 10, 12 and 13 in FIG. 2, behaves like a stub resonator. Hence, it is possible to measure real time and inline the change of dielectric properties of the polymer affinity layers 3 and 7 in FIG. 1 as a function of frequency without disturbance of the (changing) dielectric properties of surrounding water that are not directly related to the analyte of interest. This property makes the technology of the present invention unique as compared to prior art.

Preferably, the sensor is connected to an input/output (laboratory) device (spectrum analyzer or a simple computer system like e.g., the raspberry-pi), which can generate an excitation signal with a frequency in the range of 100 kHz to 3 GHz, and can be connected to a small computer platform for remotely reading-out the sensor device.

Alternatively, a frequency generator or function generator and a rectifier for measuring the amplitude of the signal can be applied. The spectrum analyzer, frequency or function generator and rectifier are operatively connected to the sensor through transmission lines. It is noted that operating frequencies of the sensor system outside the presently preferred frequency range from 100 kHz to 3 GHz are not excluded and expressly make part of the technology of the present invention.

An example of meander conductors on a PCB is shown in FIG. 2, PCB number 11. In order to ensure that all field lines go through the polymer affinity layer, the width of the meander should be limited. The width of the meander shown on PCB number 11 in FIG. 2 is most probably unacceptably high, resulting in field lines leaving the sensor and going through the water in which the sensor is submerged. A first PCB with meandering conductors as depicted in FIG. 2, PCB number 11 expressly makes part of the present invention.

It is noted that additional guard electrodes can be applied to ensure that all field lines go through the polymer affinity layer. The combination of a first PCB with meandering conductors as depicted in FIG. 2, PCB number 11 with guard electrodes, expressly makes part of the present invention.

It is noted that the straight line at the center of the PCB with number 12 (FIG. 2) may give the impression that the width of conductors 4 and 6 in FIG. 1 must be very small. However, this is not necessarily true. For example, in a first preferred embodiment of the present invention, the surface of the PCB in the center i.e., PCB 5 in FIG. 1, is completely covered with conductive material, because this will decrease the sensitivity for surface effects. Increasing the width of conductors 4 and 6 will result in a capacitance increase of the sensor and in a change of the resonant frequencies of the sensor when it is operated at high frequencies i.e., as a stub resonator. In a second preferred embodiment of the present invention, the width of conductors 4 and 6 is used as a design parameter to achieve the desired sensor properties in terms of resonant frequencies and characteristic impedance in case it is operated as a stub resonator at high frequencies.

FIG. 4 shows a cross section of a sensor similar to the sensor described in FIG. 1. The width of the conductors 22 and 28 on the PCB in the center (PCB 25) is increased as compared to the previously explained situation shown in FIG. 2, PCB number 12. An undesired effect of the increased width of conductors 22 and 28 is the increased number of electrical field lines leaving the “sandwich geometry” of the sensor and traveling through the dielectric (such as water) in which the sensor is placed, i.e. from conductor 28 through the dielectric (such as water) to conductor 31 and from conductor 22 through the dielectric (such as water) to conductor 20 respectively. In a practical application this will typically happen when the distance between the edge of the conductors 22 and 28 and the edge of the affinity layers 21 and 29 is less than about 3 times the thickness of the affinity layers 21 and 29. In case this undesired effect occurs, the sensor will become more sensitive to changes in properties of the dielectric surrounding the sensor that are not directly related to the analyte to be detected. In order to prevent this undesired effect of increasing the width of conductors 22 and 28, the sensor can be equipped with guard electrodes 23, 24, 26, 27. The width of the guard electrodes 22 and 28 shown in FIG. 4 is too small for most applications. In fact, this width is preferably at least 3 time that of the thickness of the affinity layers 21 and 29.

In the embodiment of FIG. 4, conductors 23 and 24 are galvanically separated from conductor 22, and conductors 26 and 27 are galvanically separated from conductor 28. The guard electrodes 23, 24, 26 and 27 are preferably galvanically connected forming one guard electrode. Analogous to FIG. 1, the conductors 22 and 28 on PCB 25 in FIG. 4 (the center of the sandwich) are galvanically connected and form the first sensing electrode. Hence, we have a first sensing electrode, comprising of galvanically connected conductors 22 and 28, that is guarded with a guard electrode comprising of galvanically connected conductors 23, 24, 26, 27. Similarly, galvanically connected conductors 20 and 30 are galvanically connected and form the second sensing electrode that is optionally guarded by guard electrodes 18, 19, 31 and 32. Hence we have a second sensing electrode, comprising of galvanically connected conductors 20 and 30, that is guarded with a guard electrode comprising of galvanically connected conductors 18, 19, 31, 32.

In a further preferred embodiment of the present invention, guard electrodes 18, 19, 23, 24, 26, 27, 31, 32 are all galvanically connected and grounded or held at a fixed potential and neither galvanically connected to the first sensing electrode nor to the second sensing electrode. A non limiting example of connecting the guard electrodes is to apply active guarding. With active guarding the guard electrodes 23, 24, 26 and 27 are connected with the voltage source, where the voltage is equal to that of the voltage of electrodes 22 and 28. This can be realized for instance with a unity-gain amplifier while applying negative feedback. Instead of negative feedback, active guarding can also be realized with feedforward principles, which will reduce the risk of occurrence of undesired oscillations. In a fourth preferred embodiment of the present invention, guard electrodes 18, 19, 23, 24, 26, 27, 31, 32 are all galvanically connected to the second sensing electrode, which is grounded or held at a fixed potential. It is noted that, in this embodiment, guard electrodes 18, 19, 31, 32 can be omitted and that, instead, the width of conductors 20 and 30 can be increased. An important design parameter for the sensor according to the present invention is the distance between the guard electrodes 18, 19, 23, 24, 26, 27, 31, 32 and the first and second electrodes respectively. For example, a very small distance between guard electrodes 23, 24 on one hand and conductor 22 on the other hand, will keep field lines inside the sensor but will also result in a large parasitic capacitance, decreasing the sensitivity of the sensor.

In a further embodiment of the invention a first polymer affinity layer 3 is placed on top of the first PCB and second polymer affinity layer 7 that is placed below the first PCB, see also FIG. 1. Polymer affinity layers 3 and 7 may comprise of a functionalized polymer, such as PDMS or chemically modified PDMS, designed to specifically absorb a targeted analyte.

It is noted that, although the sensor according to the present invention is very feasible to be operated as a stub resonator at high frequencies, it can also be applied for capacitance measurements at low frequencies i.e., at frequencies (far) below the lowest stub resonator resonant frequency of the sensor or, in other words, far below the base resonant frequency of the sensor in case it is applied as a quarter wave length open ended stub resonator. A typical frequency range for operating the sensor for capacitance measurements is 0 Hz (DC) to 100 kHz. Application of the sensor according to the present invention at frequencies below the lowest stub resonator resonant frequency of the sensor, expressly makes part of the present invention.

Relating to the construction materials, indicated with numbers 1, 5 and 7 in FIG. 1, may relate to printed circuit board construction material e.g., glass reinforced epoxy laminate sheets, FR4 material. It is noted that also glass, ceramics and any other (water) resistant materials are very feasible support materials for the conductors in FIG. 1.

Regarding the affinity layers 3 and 7 in FIG. 1, it is noted that besides PDMS or chemically modified PDMS, also other polymers e.g., polyethylene-co-vinylacetate are very feasible. The polymer polyethylene-co-vinylacetate is especially feasible for detection of VOCs (volatile organic compounds). Dodecyl acrylates are very feasible for the production of affinity layers selective for lead ions. For the detection of polar VOCs, polysiloxanes modified with polar units like SFXA are very feasible. The abbreviation FPOL stands for molecular structures like structure 34 in FIG. 5. The abbreviation SFXA stands for molecular structures like structure 35, 36 and 37 in FIG. 5. Sensors with an affinity layer containing at least 1 ppm of beforementioned molecules expressly makes part of the present invention.

In the following a number of non-limiting application examples of the sensor are mentioned.

In a first application the sensor is applied to detect traces of hydrophobic compounds (or metabolites thereof) like oil traces and polychlorinated biphenyls in water.

In a second application the sensor is applied to detect traces of metal ions or heavy metal ions like lead ions in water.

In a third application, the sensor is applied to detect nutrients in water such as phosphates, nitrates and sulfates.

In a fourth application, the sensor is applied to detect detergents in water including non-ionic detergents e.g., pentaerythrityl palmitate and ionic detergents e.g., sodium dodecyl sulfate.

In a fifth application the sensor is applied to detect medicine (or metabolites thereof) traces in water.

In a sixth application the sensor is applied to detect pesticides, (or metabolites thereof) in water.

In a seventh application, the sensor is applied to detect traces of drugs in water e.g., narcotics, XTC and cocaine.

It will be understood that other applications can also be envisaged in accordance with the present invention.

The present invention is by no means limited to the above described preferred embodiments thereof. The rights sought are defined by the following claims, within the scope of which many modifications can be envisaged.

1. A sensor for measuring the dielectric properties of chemical compounds and/or ions in a fluid, such as water, the sensor comprising:
a first printed circuit board (PCB) having a first side and a second side;
a first conductor on the first side of said first PCB;
a second conductor on the second side of said first PCB;
at least one galvanic connection between the first conductor and the second conductor;
a first polymer affinity layer on the first side of the first PCB;
a second polymer affinity layer on the second side of the first PCB;
a second PCB, wherein the second PCB is equipped with holes and with a first conductor plate, wherein the second PCB is placed on top of the first polymer affinity layer;
a third PCB, wherein the third PCB is equipped with holes and with a second conductor plate, wherein the third PCB is placed on top of the second polymer affinity layer;
at least one galvanic connection between the first conductor plate and the second conductor plate;
at least a first function generator that is operatively connected to the first and second conductors as well as to the first and second conductor plates; and
at least a first spectrum analyzer or rectifier that operatively connected to the first and second conductors as well as to the first and second conductor plate for measuring the amplitude of the electrical signal transmitted from the function generator through the measuring system;
wherein, in use, the sensor is placed in the fluid and fluid contacts the polymer affinity layer through the holes and wherein impurities in the fluid are selectively absorbed and dispensed in the polymer layers thereby changing the dielectric properties of the polymer affinity layers.

2. The sensor according to claim 1, further comprising at least two vias, wherein the first and second conductors are galvanically interconnected through the at least two vias on the first PCB.

3. The sensor according to claim 1, further comprising a housing with a fluid inlet and a fluid outlet in which sensor parts are placed and wherein, in use, a fluid flow is provided in the housing, wherein the sensor is at least partially submerged in the fluid.

4. The sensor according to claim 1, wherein the first and second conductors of the first PCB comprise a meandering pattern over the PCB surface, thereby increasing the effective length of said conductors.

5. The sensor according to claim 1 further comprising guard electrodes.

6. The sensor according to claim 5, wherein a width of the guard electrodes is at least three times a thickness of the affinity layers.

7. The sensor according to claim 5, wherein the guard electrodes comprise galvanically connected electrodes, and wherein the guard electrodes are preferably grounded.

8. The sensor according to claim 5, wherein the guard electrodes are held at a fixed potential and are not galvanically connected to the electrodes.

9. The sensor according to claim 5, wherein the guard electrodes and the conductors are spaced apart at a predetermined distance.

10. The sensor according to claim 1, wherein the sensor is configured for measuring the dielectric losses in the first and second polymer affinity layers.

11. The sensor according to claim 1, wherein the sensor is configured for one or more of: detection of oil traces in water, detection of metal ions in water, detection of medicine traces in water, detection of pesticides in water, detection of traces of drugs in water e.g., narcotics, XTC and cocaine, detection of detergents in water including non-ionic detergents e.g., pentaerythrityl palmitate and ionic detergents e.g., sodium dodecyl sulfate, and for detection of nutrients in water such as phosphates, nitrates and sulfates.

12. The sensor according to claim 1, wherein one or more of the polymer affinity layers comprises a functionalized polymer, wherein the functionalized polymer is configured to absorb a specific analyte.

13. A system for detecting substances and/or analytes in a fluid, the system comprising:
a housing having a fluid inlet and a fluid outlet; wherein the housing, in use, is configured to be at least partially filled with fluid and wherein, in use, a fluid flow is present between the fluid inlet and the fluid outlet; and
a sensor comprising:
a first printed circuit board (PCB) having a first side and a second side;
a first conductor on the first side of said first PCB;
a second conductor on the second side of said first PCB;
at least one galvanic connection between the first conductor and the second conductor;
a first polymer affinity layer on the first side of the first PCB;
a second polymer affinity layer on the second side of the first PCB;
a second PCB, wherein the second PCB is equipped with holes and with a first conductor plate, wherein the second PCB is placed on top of the first polymer affinity layer;
a third PCB, wherein the third PCB is equipped with holes and with a second conductor plate, wherein the third PCB is placed on top of the second polymer affinity layer;
at least one galvanic connection between the first conductor plate and the second conductor plate;
at least a first function generator that is operatively connected to the first and second conductors as well as to the first and second conductor plates; and
at least a first spectrum analyzer or rectifier that operatively connected to the first and second conductors as well as to the first and
second conductor plate for measuring the amplitude of the electrical signal transmitted from the function generator through the measuring system,
wherein the sensor is positioned in the housing between the fluid inlet and the fluid outlet, and wherein, during use, the sensor is at least partially submerged in the fluid.

14. A method for measuring dielectric properties of chemical compounds and/or ions in water, comprising the step of providing a sensor comprising:
a first printed circuit board (PCB) having a first side and a second side;
a first conductor on the first side of said first PCB;
a second conductor on the second side of said first PCB;
at least one galvanic connection between the first conductor and the second conductor;
a first polymer affinity layer on the first side of the first PCB;
a second polymer affinity layer on the second side of the first PCB;
a second PCB, wherein the second PCB is equipped with holes and with a first conductor plate, wherein the second PCB is placed on top of the first polymer affinity layer;
a third PCB, wherein the third PCB is equipped with holes and with a second conductor plate, wherein the third PCB is placed on top of the second polymer affinity layer;
at least one galvanic connection between the first conductor plate and the second conductor plate;
at least a first function generator that is operatively connected to the first and second conductors as well as to the first and second conductor plates; and
at least a first spectrum analyzer or rectifier that operatively connected to the first and second conductors as well as to the first and second conductor plate for measuring the amplitude of the electrical signal transmitted from the function generator through the measuring system,
wherein, in use, the sensor is placed in the fluid and fluid contacts the polymer affinity layer through the holes and wherein impurities in the fluid are selectively absorbed and dispensed in the polymer layers thereby changing the dielectric properties of the polymer affinity layers.

15. A method according to claim 14, further comprising the step of operating at a frequency of the first function generator below its quarter wave length open ended stub resonator resonant frequency.

16. The method according to claim 14, further comprising the step of operating at a frequency of the first function generator below 100 kHz.

17. The method according to claim 14, the method comprising detecting one or more of: oil traces in water, metal ions in water, medicine traces in water, pesticides in water, traces of drugs in water e.g., narcotics, XTC and cocaine, detergents in water including non-ionic detergents e.g., pentaerythrityl palmitate and ionic detergents e.g., sodium dodecyl sulfate, and of nutrients in water such as phosphates, nitrates and sulfates.

18. The method according to claim 14, the method comprising measuring dielectric losses in the first and second polymer affinity layer.

19. The method according to claim 15, further comprising the step of operating at a frequency of the first function generator below 100 kHz, and further comprising measuring dielectric losses in the first and second polymer affinity layer.

20. The sensor according to claim 2, further comprising a housing with a fluid inlet and a fluid outlet in which sensor parts are placed and wherein, in use, a fluid flow is provided in the housing, wherein the sensor is at least partially submerged in the fluid, and wherein the first and second conductors of the first PCB comprise a meandering pattern over the PCB surface, thereby increasing the effective length of said conductors.
